(19) United States
(12) Patent Application Publication
(10) Pub. No.: US 2019/0038172 A1
(43) Pub. Date: Feb. 7, 2019

(54) FAST GROUP-WISE TECHNIQUE FOR DECOMPOSING GSR SIGNALS ACROSS GROUPS OF INDIVIDUALS

(71) Applicant: THOMSON Licensing, Issy-les-Moulineaux (FR)

(72) Inventors: Brian Charles ERIKSSON, San Jose, CA (US); Swayambhoo Jain, Sunnyvale, CA (US); Urvashi Oswal, Madison, WI (US); Kevin Shuai Xu, Toledo, OH (US)

(21) Appl. No.: 15/758,966

(22) PCT Filed: Nov. 12, 2015
(86) PCT No.: PCT/US2015/060359

§ 371 Date: Mar. 9, 2018

Related U.S. Application Data
(60) Provisional application No. 62/216,080, filed on Sep. 9, 2015.

(51) Int. Cl. A61B 5/053; A61B 5/00

(52) U.S. Cl. CPC A61B 5/0533; A61B 5/165; A61B 5/7271; A61B 5/7203

(57) ABSTRACT

A method for correlating Galvanic Skin Response (GSR) signals from multiple users watching the same content to identify scenes of interest in such content includes filtering GSR signals from each user by subtracting consecutive GSR signal samples from each other. The user reaction portion and baseline portion of the GSR signals for the users are collectively optimized to recover non-zero user responses for the users. The locations in the content having the non-zero responses for the users are then identified as the scenes of interest in such content for the users.

### CROSS-REFERENCE TO RELATED APPLICATIONS

This application claims priority under 35 U.S.C. 119(e) to U.S. Provisional Patent Application Ser. No. 62/216,080, filed Sep. 9, 2015, the teachings of which are incorporated herein.

### TECHNICAL FIELD

This invention relates to a technique for correlating the Galvanic Skin Response (GSR) of multiple individuals watching the same content.

### BACKGROUND ART

Galvanic Skin Response (GSR) signals constitute a measure of skin conductance. Measurement of GSR occurs by placing two electrodes on the skin of a user, then applying very small voltage across the electrodes and measuring the current passing through the skin. More current means higher conductance, thus establishing the GSR signal. Due to evolution, human beings sweat whenever they see something exciting or simulating. The sweat contains electrolytes which increase the conductance of the skin. Thus, while a user watches content, the GSR signal will increase as the user sees something stimulating in the content.

In connection with group viewing of the same content, having some measure of the GSR signals for the group will prove useful. However, past attempts to combine GSR signals from individual users has not yielded satisfactory results.

Thus a need exists for a technique for combining GSR signals from multiple users.

### BRIEF SUMMARY

Briefly, a method for correlating Galvanic Skin Response (GSR) signals from multiple users watching the same content to identify scenes of interest in such content includes filtering GSR signals from each user by subtracting consecutive GSR signal samples from each other. The user reaction portion and baseline portion of the GSR signals for the users are collectively optimized to recover non-zero user responses for the users. The locations in the content having the non-zero responses for the users are then identified as the scenes of interest in such content for the users.

### BRIEF SUMMARY OF THE DRAWINGS

FIG. 1 depicts a setting where multiple users observe the content;

FIG. 2 depicts the Galvanic Skin Response (GSR) Signals from four users watching content as depicted in FIG. 1;

FIG. 3 depicts different components of the GSR signal from one of the users of FIG. 2;

FIG. 4 depicts a block diagram of system for processing each user's GSR signals; and FIG. 5 depicts a method for correlating GSR signals from multiple users in accordance with the present principles.

### DETAILED DISCLOSURE

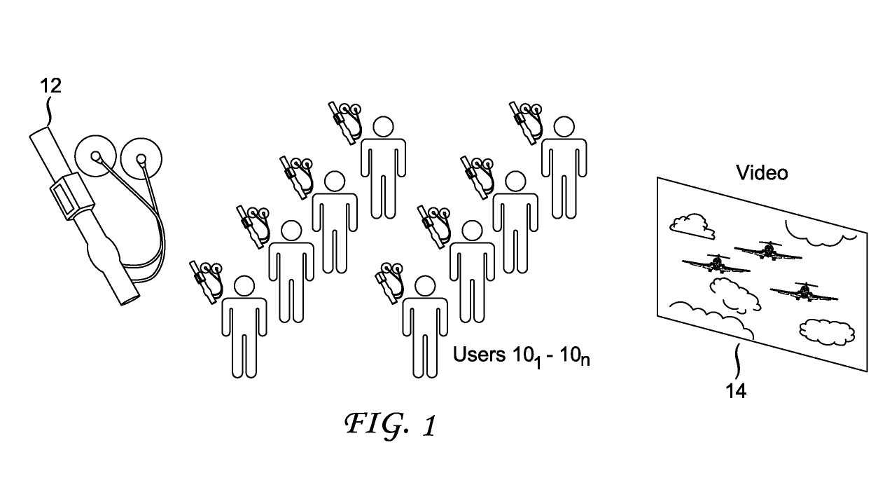

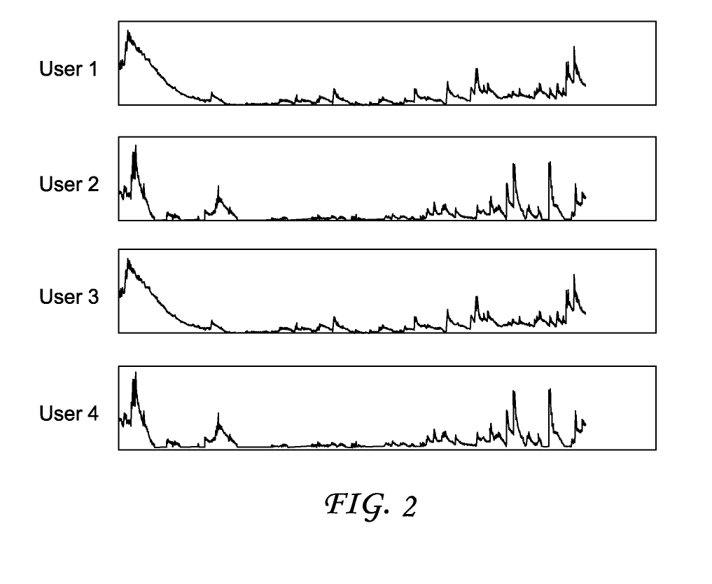

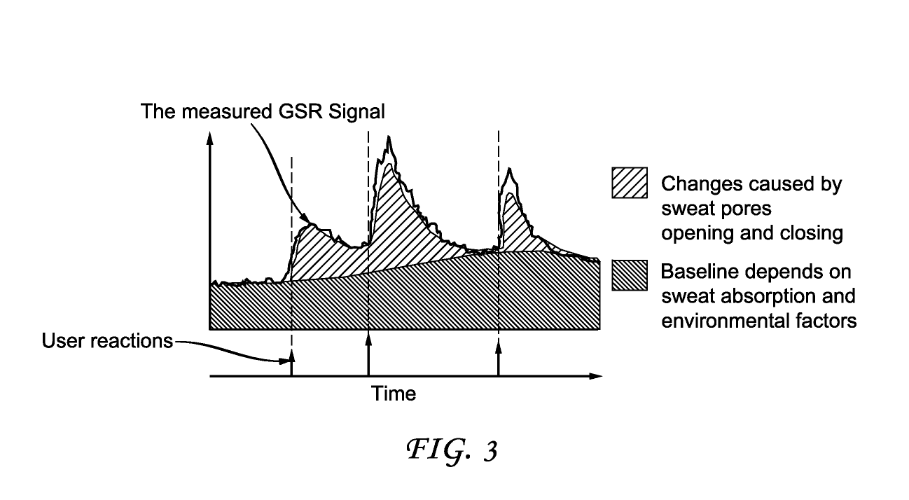

To understand the problem of correlating GSR signals from multiple users, consider the setting depicted in FIG. 1 where a group of users 10₁-10ₙ (wherein n is an integer greater than 0) watch the same content. Each user 10ᵢ (where i is an integer≤n) wears a device 12 (e.g., a watch), which wirelessly synchronizes with all the other wearable devices in the group. Each of these wearable devices 12 has electrodes which measure the user's GSR signal as the user watches the same content with the other users. The GSR signals for four users commonly observing a toy scene displayed on the display device 14 in FIG. 1 appears in FIG. 2. Since the users 10₁-10ₙ of FIG. 1 all watch the same content, a correlation exits between the GSR signals observed across various users as seen in FIG. 2. The GSR signal processing technique of present principles exploits this correlation across the users to find the instances in the content that are of interest to all of the users 10₁-10ₙ.

Even though a correlation exists between the users' GSR signals and the content watched, extracting the exact moment when each user becomes stimulated has proven problematic. There are three reasons for this. (1) The exact relationship between the moments of interest in the content and the increase in each user's GSR signal is not straight forward. (2) The increase in the GSR signal due each user's stimulation to the viewed content lies on top of an already existing but unknown baseline GSR signal. This unknown baseline signal depends on related environmental factors like temperature, rate of absorption etc. (3) Some sensor noise always exists in the GSR signal which corrupts the GSR signal further.

As discussed, a correlation exists between an individual user's GSR and the content observed by that user. In addition, when the group of users watches the same content, a correlation exists across the various users GSR measurements as well. The GSR signal processing technique of the present principles exploits these correlations to find moments (scenes) of group interest in the content.

In order to better understand the GSR signal processing technique of the present principles, consider the following notations. Suppose the content is of duration ‘T’ seconds and sampling occurs every second. Let ‘xᵢ’ be a ‘T’ dimensional vector representing the i^{th} user's reactions to the content every second, and ‘hᵢ’ be a ‘t’ dimensional vector(with t<<T) capturing the typical sweat response of the i^{th} user. Each user is modeled as a Linear Time Invariant (LTI) system, with an impulse response ‘hᵢ’ representing the typical way each user sweats when the user finds something exciting in the content.

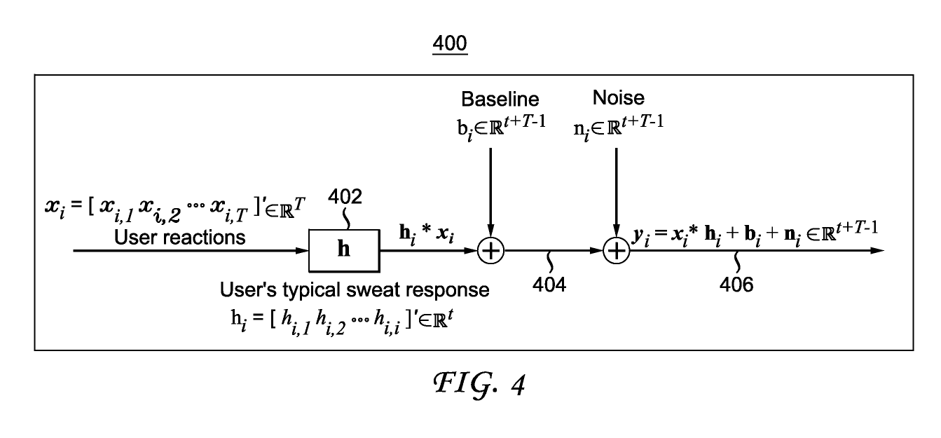

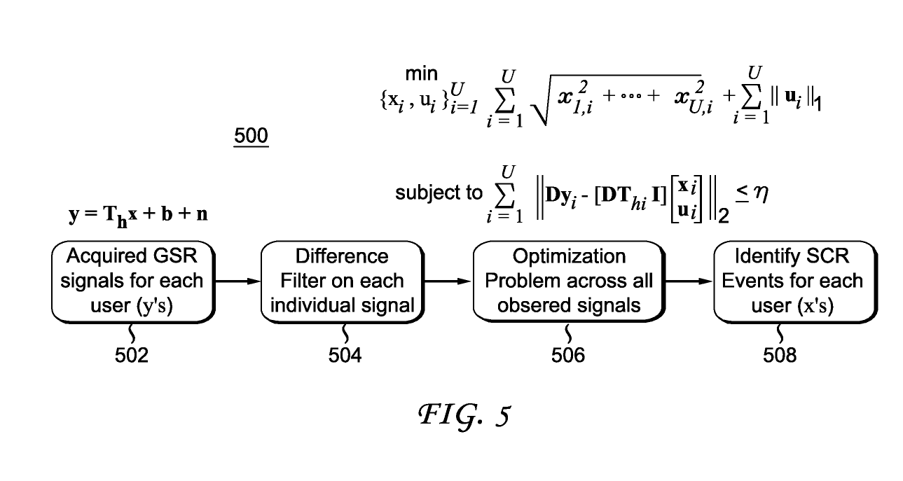

FIG. 4 depicts a block schematic diagram of a system 400 for obtaining fine grain GSR signal responses for an individual user in accordance with an illustrative embodiment of the present principles. The system 400 typically comprises a processor or computer that typically includes a central processing unit (CPU) (not shown) along with various peripheral devices (keyboard, mouse, display, network adapters) (not shown) along with a power supply (not shown). As described in greater detail hereinafter, the system 400 of FIG. 4 advantageously accounts for the GSR baseline signal ‘b’ and noise as ‘n’ in determining an individual user's GSR response.

In FIG. 4, ‘the system 400 includes a block 402 that performs the convolution of ‘x’ and ‘h’, represented by the term x*h′. The convolution operation performed by the block 402 can be represented as a matrix vector multiplication as follows:

$$y = h * x = \underbrace{{[ \begin{matrix}{h}_{1} & 0 & … & 0 \\ {h}_{2} & {h}_{1} & ⋮ & ⋮ \\ ⋮ & ⋮ & ⋱ & 0 \\ {h}_{t} & {h}_{t - 1} & ⋮ & {h}_{1} \\ 0 & {h}_{t} & & \\ ⋮ & ⋮ & ⋱ & \\ 0 & … & … & {h}_{t}\end{matrix} ]}^{( t + T - 1 ) \times T Tall Toeplitz Matrix}}_{{T}_{h}} [ \begin{matrix}{x}_{1} \\ ⋮ \\ {x}_{T}\end{matrix} ]$$

where the ‘Tₕ’ is a (t+T−t) by T tall Toeplitz matrix as shown above. With this the final observation ‘y’ can be written as

y=x*h+b+n=Tₕx+b+n

In accordance with an aspect of the present principles, the effect of the baseline signal is mitigated by filtering the observed signal for each user such that the baseline component of the GSR signal does not obfuscate the user's response. Such filtering occurs by subtracting consecutive components from observed GSR signal ‘y’ via block 404 in the system 200 in the following manner:

$$y = {T}_{h} x + b + n {D = [ \begin{matrix}1 & - 1 & 0 & … & 0 \\ 0 & 1 & - 1 & … & 0 \\ ⋮ & ⋱ & ⋱ & … & 0 \\ 0 & … & … & 1 & - 1\end{matrix} ]}^{} Dy = {DT}_{h} x + Db + Dn$$

The subtraction of consecutive samples of the observation can be achieved simply by multiplying the observation by the difference matrix ‘D’ shown above. The above-described matrix includes noise subtraction performed by the block 206.

After taking the difference of the consecutive samples the observations, the user reactions ‘xᵢ’ part in the GSR signal and the transformed baseline ‘Dbᵢ’ component of the GSR signal have same structure. Both ‘xᵢ’ and ‘Dbᵢ’ are sparse. Since users are watching the same content, the vectors ‘xᵢ’ are non-zero at same locations. This problem is solved the following optimization

$${min}_{{\{ {x}_{i} , {u}_{i} \}}_{i = 1}^{U}} {\sum }_{i = 1}^{U} \sqrt{{x}_{1 , i}^{2} + … + {x}_{U , i}^{2}} + {\sum }_{i = 1}^{U} {‖ {u}_{i} ‖}_{1}$$

$$subject to {\sum }_{i = 1}^{U} {‖ {Dy}_{i} - [ \begin{matrix}{DT}_{{h}_{i}} & I\end{matrix} ] [ \begin{matrix}{x}_{i} \\ {u}_{i}\end{matrix} ] ‖}_{2} \leq η$$

where xᵢ represents the response of a user uᵢ represents, uᵢ=Dbᵢ represents filtered baseline signal for user uᵢ, Dyᵢ represents filtered observation for user uᵢ, D represents a difference matrix and Thᵢ represents Toeplitz matrix for user typical ui sweat response and I represents identity matrix.

The parameter ‘η’ is the tuning parameter used to fine-tune the output. Standard open source numerical optimization software packages can be used to solve this problem easily. The solution of above problem is such that the recovered ‘xᵢ’ have non-zeros at similar locations. This gives us the points of group interest in the visual content.

In contrast to prior approaches, the GSR correlation technique of the present principles makes use of a more realistic single model for the GSR observations and considers the correlations of groups of users watching the same content. In addition, the signal model of the present principles yields an optimization problem that is much easier to solve than prior approaches. For example, prior approaches require many lines of code, while the technique of the present principles can be implemented in 4 lines of code. In addition, the computation time required by this invention is significantly faster than the previous approach.

FIG. 5 depicts a flow chart of a method 500 in accordance with the present principles for correlating GRS signals from multiple users. The first step is to acquire GSR signals for each user during step 502. Next, the individual user's GSR signals are filter to subtract consecutive samples during step 504 (typically using the system 400 of FIG. 4). The reaction portion and baseline portion of the GSR signals for the users are optimized during step 506 to recover non-zero user responses at similar locations in the content. The optimization could be performed by the system 400 or another processor. The similar locations in the content having the non-zero responses are then identified as the scenes of interest in such content during step 508.

The foregoing describes a technique for correlating GSR signals of multiple users.

1. A method for correlating Galvanic Skin Response (GSR) signals from multiple users watching the same content to identify scenes of interest in such content, comprising:
filtering GSR signals from each user by subtracting consecutive GSR signal samples from each other;
collectively optimizing a user reaction portion and baseline portion of the GSR signals for the users to recover non-zero user responses for the users during content viewing; and
identifying locations in the content having the non-zero responses for the users as scenes of interest in such content for the users.

2. The method according to claim 1 wherein the filtering step includes removing noise in the GSR signal.

3. The method according to claim 1 wherein the filtering step includes multiplying GSR signal samples by a difference matrix.

4. The method according to claim 1 wherein the optimization occurs by solving the following:
$${min}_{{\{ {x}_{i} , {u}_{i} \}}_{i = 1}^{U}} {\sum }_{i = 1}^{U} \sqrt{{x}_{1 , i}^{2} + … + {x}_{U , i}^{2}} + {\sum }_{i = 1}^{U} {‖ {u}_{i} ‖}_{1}$$
$$subject to {\sum }_{i = 1}^{U} {‖ {Dy}_{i} - [ \begin{matrix}{DT}_{{h}_{i}} & I\end{matrix} ] [ \begin{matrix}{x}_{i} \\ {u}_{i}\end{matrix} ] ‖}_{2} \leq η$$
where xi represents the response of a user uᵢ, uᵢ=Dbᵢ represents filtered baseline signal for user uᵢ, Dyᵢ represents filtered observation for user uᵢ, D represents a difference matrix and Thᵢ represents Toeplitz matrix for user typical uᵢ sweat response and I represents identity matrix.

5. A system for achieving fine grain response of a Galvanic Skin Response (GSR) signals from a user while the user watches content to identify scenes of interest in such content, comprising:
a processor configured to (a) filter GSR signals from each user by subtracting consecutive GSR signal samples from each other; (b) collectively optimize a user reaction portion and baseline portion of the GSR signals for the users to recover non-zero user responses for the users during content viewing; and (c) identify locations in the content having the non-zero responses for the users as scenes of interest in such content for the users.

6. The system according to claim 5 wherein the filtering includes removal of noise in the GSR signal.

7. The system according to claim 5 wherein the filtering occurs by multiplying incoming samples by a difference matrix.

8. The system according to claim 5 wherein the processor performs optimization by solving the following:
$${min}_{{\{ {x}_{i} , {u}_{i} \}}_{i = 1}^{U}} {\sum }_{i = 1}^{U} \sqrt{{x}_{1 , i}^{2} + … + {x}_{U , i}^{2}} + {\sum }_{i = 1}^{U} {‖ {u}_{i} ‖}_{1}$$
$$subject to {\sum }_{i = 1}^{U} {‖ {Dy}_{i} - [ \begin{matrix}{DT}_{{h}_{i}} & I\end{matrix} ] [ \begin{matrix}{x}_{i} \\ {u}_{i}\end{matrix} ] ‖}_{2} \leq η$$
where xᵢ represents the response of a user uᵢ, uᵢ=Dbᵢ represents filtered baseline signal for user uᵢ, Dyᵢ represents filtered observation for user uᵢ, D represents a difference matrix and Thᵢ represents Toeplitz matrix for user typical uᵢ sweat response and I represents identity matrix.

9. A system for achieving fine grain response of a Galvanic Skin Response (GSR) signals from a user while the user watches content to identify scenes of interest in such content, comprising:
a filter for filtering GSR signals from each user by subtracting consecutive GSR signal samples from each other;
an optimizer for collectively optimizing a user reaction portion and baseline portion of the GSR signals for the users to recover non-zero user responses for the users during content viewing; and
an identifier for identifying locations in the content having the non-zero responses for the users as scenes of interest in such content for the users.
